(19) United States
(12) Patent Application Publication
(10) Pub. No.: US 2024/0350815 A1
(43) Pub. Date: Oct. 24, 2024

(54) RECHARGEABLE ELECTRONIC IMPLANT

(71) Applicants: Walter MEHNERT, Ottobrunn (DE); Thomas THEIL, Feldafing (DE)

(72) Inventors: Walter MEHNERT, Ottobrunn (DE); Thomas THEIL, Feldafing (DE)

(21) Appl. No.: 18/685,582

(22) PCT Filed: Aug. 11, 2022
(86) PCT No.: PCT/EP2022/072611

§ 371 Date: Feb. 22, 2024

(30) Foreign Application Priority Data
Aug. 25, 2021 (DE) 10 2021 209 355.8
Sep. 17, 2021 (DE) 10 2021 210 373.1
Oct. 18, 2021 (DE) 10 2021 126 954.7
Mar. 17, 2022 (WO) PCT/EP2022/057089

(51) Int. Cl. A61N 1/375; A61N 1/378

(52) U.S. Cl. CPC A61N 1/3756; A61N 1/37512; A61N 1/3787

(57) ABSTRACT

The application relates to an electronic implant for implantation into a body of a living being and for monitoring a bodily function, in particular a pacemaker for monitoring and controlling the bodily function, the implant including: an electrode portion that is, according to its intended purpose, to be attached to or to be arranged at a body portion; an electronics assembly connected to the electrode portion, which is configured to monitor at least the bodily function via the electrode portion; an energy storage to supply the electronics assembly with electrical energy which can be recharged with electrical energy after discharge; and an energy receiving portion electrically connected to the energy storage, which is configured so as to be able to receive energy without contact and to deliver the energy to the energy storage for recharging the energy storage; wherein the energy receiving portion includes: a coil extending along a coil axis and being configured to receive the energy and to deliver the energy to the energy storage when passed through by an external alternating magnetic field, a core, which is located in the coil and extends along the coil axis, and at least one field collector, which is located at one end of the core in the direction of the coil axis and has larger dimensions transverse to the coil axis than the core.

The invention relates to an electronic implant for implantation into a body of a living being and for monitoring a bodily function, in particular a pacemaker for monitoring and controlling the bodily function. In particular, the invention relates to an electronic pacemaker which is intended to be implanted into/on the human heart.

An electronic implant for implantation into the human heart is known from patent publication WO 2012/013212 A1. The implant described there communicates with a higher-level control unit by radio, wherein the implant together with the control unit assumes the functions of a pacemaker, a defibrillator and a unit for recording cardiac activity, such as a cardiogram.

The control unit supplies the implant with electrical energy so that the implant, in its function as a pacemaker/defibrillator, can emit voltage pulses to stimulate the heart or detect body signals to record cardiac activity. The electrical energy is transmitted by the control unit emitting an alternating electromagnetic field and the implant receiving the corresponding energy by induction. For this purpose, the implant has a receiving coil with a core.

However, the type of energy transfer described in the patent document is extremely problematic in this field of application. The reason for this is that there are opposing effects in this field of application.

In general, it would be desirable to set the frequency of the alternating electromagnetic field as high as possible. However, this does not make sense because the field does not penetrate far enough into the human body with increasing frequency due to skin effects and does not reach the implant, and the increasing AC resistance reduces the charging current.

If an attempt is made to counter this effect by reducing the frequency while increasing the number of turns of the receiving coil, the problem arises that the AC resistance of the receiver coil increases quadratically with the number of turns and limits the energy transfer due to voltage drops.

The core shown only slightly eliminates the above problems.

Overall, energy transmission in this field of application has not been solved satisfactorily, if at all.

Against the above background, the object of the invention is to create an implant that permits a small volume, low weight and improved energy transmission. At least it is an object of the invention to create an alternative implant.

This object(s) is solved by an implant according to patent claim 1. Preferred embodiments are subject-matters of the dependent claims.

The electronic implant for implantation into a body of a living being and for monitoring a bodily function, in particular a pacemaker for monitoring and controlling the bodily function, comprises:
an electrode portion that is, according to its intended purpose, to be attached to or to be arranged at a body portion;
an electronics assembly connected to the electrode portion, which is configured to monitor at least the bodily function via the electrode portion;
an energy storage to supply the electronics assembly with electrical energy which can be recharged with electrical energy after discharge; and
an energy receiving portion electrically connected to the energy storage, which is configured so as to be able to receive energy without contact by induction and to deliver the energy to the energy storage for recharging the energy storage; wherein
the energy receiving portion comprises:
a coil extending along a coil axis and being configured to receive energy and deliver the energy to the energy storage when passed through by an external (outside the body) generated alternating magnetic field,
a core, which is located in the coil and extends along the coil axis, the coil being preferably wound on and around the core, and
at least one field collector, which is located at one end of the core in the direction of the coil axis and has larger dimensions transverse to the coil axis than the core.

The implant is, for example, a cardiac pacemaker, a brain pacemaker, an organ pacemaker or an analysis unit. The latter analysis unit is designed, for example, to determine parameters such as blood pressure and/or blood values and/or record a cardiogram continuously or at certain intervals. Particularly preferably, the implant is a single-chamber pacemaker or part of a multi-chamber pacemaker network that is located in the human heart or on the human heart or is implanted in these positions. The pacemaker network, for example, has two or three implants connected via electrical signals, each being implanted into a ventricle, anchored there and in communication with each other.

Depending on the purpose of the implant, the electrode portion contains a certain number of electrodes, with one of the electrodes acting as a ground.

If the implant assumes the function of one of the pacemakers mentioned, the electrodes are intended to be connected to or to be attached to the body portion to be stimulated, for example the heart or brain.

In general, the electrodes mentioned may be cable electrodes, for example. In particular, the implant preferably comprises in this context one cable of a certain length per cable electrode which cable, according to the intended purpose, may be guided to a desired area of the body portion within the body. A preferably spiral segment used to anchor the cable electrode in the area of the body portion is formed at the end of the cable.

Alternatively, the electrode portion can do without cable electrode(s) as well. In this case, said electrodes are formed on an outer surface of the implant, wherein it is implanted so that the electrodes can touch an area of the body portion and/or be anchored there. This configuration is particularly advantageous if the implant is the pacemaker or part of the pacemaker network, each of which is to be implanted completely into/on the heart. The pacemaker network therefore contains several implants according to the invention with corresponding electrode portions that are exposed on the outer surface of the respective units.

In a further alternative, the electrode portion may be composed of a combination of at least a single cable electrode and of at least a single electrode formed on the outer surface. In this case, the implant is preferably arranged at the body portion so that the electrode formed on the outer surface comes in contact with the corresponding area of the body portion and/or is anchored there. The other electrode, i.e., the cable electrode, is guided to another area of the body portion and anchored or attached there.

The electronics of the implant according to the invention are set up to monitor at least one or more bodily functions. This includes, for example, the functions of the aforementioned analysis unit, i.e. the recording of data, for example from a cardiogram or the recording of blood values.

If the implant is the said pacemaker or part of the pacemaker network to be implanted into/on the human heart, the electronics assembly is configured to monitor the heartbeat and, based on this, recognize whether the heartbeat needs to be controlled. If this is the case, the electronics assembly generates a stimulation pulse, in particular a voltage pulse, and transmits this to the body portion via the electrode portion.

With regard to the structure and functions of the pacemaker network, reference is made to the explanations in patent application EP 3756726 A2. In particular, paragraphs [0011-0028] of EP 3756726 A2 are incorporated by reference.

The energy storage of the implant according to the invention is preferably an electrochemical accumulator that can be recharged, in particular a lithium-ion accumulator. The energy storage is preferably dimensioned in such a way that it can supply the entire implant with electrical energy for a service life of 0.5, 0.6, 0.7, 0.8, 0.9, 1.0, 1.1, 1.2, 1.3, 1.4, 1.5, 1.6, 1.7, 1.8, 1.9, 2.0 years. For example, the energy storage has a charging capacity of 400 ampere seconds As (coulombs).

The energy storage may comprise a plurality of energy storage units which are distributed and arranged separately from each other at different positions in the implant, wherein at least one or each of the energy storage units is preferably an electrochemical accumulator unit, in particular a lithium-ion accumulator unit.

Preferably, the energy receiving portion comprises at least one rectifier and at least one capacitor, which are located between the coil and the energy storage. The coil transfers the received energy to the capacitor via the rectifier. In this context, the charging (alternating) current (AC) emitted by the coil is rectified by the rectifier and fed to the energy storage by the capacitor.

The structure of the energy receiving portion is of key importance, with the field collector (and preferably the core) being the important element here.

The energy receiving portion is configured to receive the energy by induction, for which purpose it comprises a coil through which the external alternating magnetic field passes. Depending on the change in the magnetic flux passing through it, the coil generates the corresponding charging voltage and a corresponding charging current flow via the rectifier, which is used to recharge the energy storage. In other words, the charging voltage is proportionally dependent on the frequency and amplitude of the magnetic flux of the alternating magnetic field. In particular, the core and the field collector are not an element, such as a Wiegand wire/pulse wire, which shows a large Barkhausen jump, in the form of a Bloch wall running over the wire, when the magnetic field changes by a certain amplitude and therefore induces pulses of the same level in the coil regardless of the frequency of the alternating magnetic field. In general, the material of the core as a magnetic flux conductor and the material of the field collector have irregularly magnetically aligned domains.

The coil axis of the coil preferably defines the orientation of the implant.

To increase the magnetic flux through the coil, the energy receiving portion on the one hand comprises the core, which is arranged along the coil axis within the coil, and on the other hand the field collector.

According to the invention, the field collector has larger dimensions than the core transverse, in particular perpendicular, to the coil axis and is arranged at one end of the core in the direction of the coil axis.

The external alternating electromagnetic field (B₀) is preferably generated in such a way that it is aligned in the direction of the coil axis, i.e. the B vector points in the direction of the coil axis. Due to its larger dimensions, the field collector ensures that the electromagnetic alternating field is increasingly conducted into the core via a larger field collection area. In other words, the field collector ensures that the magnetic flux density within the core and thus within the coil—n×B₀—increases considerably.

A cross-sectional area of the field collector perpendicular to the coil axis is preferably 1.5-, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 times larger than a cross-sectional area of the core perpendicular to the coil axis.

A charging device preferably generates the external alternating electromagnetic field, preferably with a magnetic flux density of B₀=0.5 mT, 1 mT, 2 mT, 3 mT, 4 mT, 5 mT, 6 mT, 7 mT, 8 mT, 9 mT, 10 mT, 11 mT, 12 mT, 13 mT, 14 mT, 15 mT, 16 mT, 17 mT, 18 mT, 19 mT, 20 mT, 21 mT, 22 mT, 23 mT, 24 mT, 25 mT, 26 mT, 27 mT, 28 mT, 29 mT, 30 mT and a frequency f=0.5 kHz, 1 kHz, 2 kHz, 3 kHz, 4 kHz, 5 kHz, 6 kHz, 7 kHz, 8 kHz, 9 kHz, 10 kHz, 11 kHz, 12 kHz, 13 kHz, 14 kHz, 15 kHz, 16 kHz, 17 kHz, 18 kHz, 19 kHz, or 20 kHz. In particular, the magnetic flux density B₀ is intended to be present in the spatial area of the implanted implant, for example on the heart of a person in whom the implant is located in the function of the pacemaker on/in the heart.

The field collector amplifies the magnetic flux density n limited by the saturation field strength within the core, wherein n>=50, 60, 70, 80, 90, 100, 110, 120, 130, 140, 150, 160, 170, 180, 190, 200, 250, 300. This results in (amplification 300) a core flux density B_{C} of 1.5 T prevailing within the core with a spatially homogeneous magnetic flux density of, for example, B₀=5 mT in the vicinity of the coil. The resulting values of the charging voltage/current are sufficient to supply the energy storage with sufficient energy for recharging, even if the frequency f of the generated alternating magnetic field is in the low ranges mentioned, for example at 2 KHz.

The coil has, for example, W windings, wherein W=50, 100, 200, 300, 400, 500, 600, 700 or 800 is preferred. The winding formed by the W windings may be single-layered or multi-layered. The metal wire forming the W windings is made, for example, of copper or preferably of the lighter metal aluminum and has a wire diameter of, for example, 60 μm, 70 μm, 80 μm, 90 μm, 100 μm, 150 μm, 200 μm and a circular or rectangular cross-section. A length of the coil 6 preferably corresponds to that of the core 7, so that ends of the coil preferably correspond to ends of the core.

Preferably, the field collector is part of the core, in particular monolithic with the core and is formed from the same material. The material of the field collector and core is preferably a homogeneous magnetic material, for example a ferromagnetic or ferrimagnetic material.

Alternatively, the field collector may be an element separate from the core. Similarly, the material of the core and/or the field collector may be a homogeneous magnetic material, for example a ferromagnetic or ferrimagnetic material, even if they are separate.

For example, the field collector preferably having a cylindrical shape extends 1 mm to 5 mm in the direction of the coil axis.

The implant preferably further comprises:
a further field collector, which is located in the direction of the coil axis at another end of the core and has larger dimensions transverse to the coil axis than the core.

Preferably, the further collector is part of the core, in particular monolithic with the core and is formed from the same material.

If the field collector, the core and the further field collector are monolithic, the material is preferably the aforementioned homogeneous magnetic material, for example the ferromagnetic or ferrimagnetic material.

Alternatively, the further field collector may be a separate element from the core.

In the same way, the material of the core and/or the further field collector may also be a homogeneous magnetic material, for example a ferromagnetic or ferrimagnetic material.

For example, the further field collector preferably having a cylindrical shape extends 1 mm to 5 mm in the direction of the coil axis.

The core with the field collector and/or the further field collector preferably fully utilizes an interior space of a housing of the implant, i.e. the outer contour of the implant is defined by the core with field collector(s). Preferably, the housing accommodates all elements of the implant, namely the electronics assembly, the energy storage and the energy receiving portion. Merely the electrode portion preferably penetrates the housing and is located outside the housing. The housing is made of a non-ferromagnetic material, for example.

The coil is preferably wound around the core between the field collector and the further field collector.

The energy storage of the implant comprises at least an energy storage unit and preferably at least a further energy storage unit. The energy storage unit and, if preferably provided, the further energy storage unit is/are preferably arranged in the direction of the coil axis (SA) relative to the core.

For example, the energy storage unit and, if preferably provided, the further energy storage unit is/are arranged in the direction of the coil axis relative to the core in such a way that they are located next to the end of the core or, if one of the field collectors is arranged there, they are located on the side of the corresponding field collector facing away from the core. Preferably, the end of the core/field collector and the corresponding energy storage unit are in contact.

Preferably, one energy storage unit and the other energy storage unit are provided, with one of the energy storage units being arranged on one side of the core in the direction of the coil axis and the other of the energy storage units being arranged on the other side of the core. In other words, the energy storage units are preferably arranged in the direction of the coil axis in such a way that the core and preferably the field collector(s) are located between the energy storage units.

Particularly preferably, the energy storage of the implant comprises at least an energy storage unit and preferably at least a further energy storage unit, wherein
the energy storage unit and, if preferably provided, the further energy storage unit each have a housing which acts as the field collector and/or the further field collector.

Particularly preferably, the energy storage of the implant comprises at least an energy storage unit and preferably at least a further energy storage unit, wherein
the field collector and, if preferably provided, the further field collector has/have a recess in which the energy storage unit and, if preferably provided, the further energy storage unit is/are accommodated.

In a sectional view, which corresponds to a sectional plane in which the coil axis lies, the recess preferably has a C or U shape.

Particularly preferably, the field collector and the further field collector are provided and both preferably have the aforementioned recess, in each of which one of the energy storage units is accommodated.

The energy storage units are preferably accommodated in the respective recess in such a way that they are either completely accommodated/recessed in the corresponding recess or protrude from an end face of the corresponding field collector. In the latter case, a housing of the respective energy storage unit is preferably formed from the following materials of the core/field collectors. A cross-sectional shape of the housing of the respective energy storage unit is preferably an exact fit with a cross-sectional shape of the recess.

Preferably, the implant is designed in such a way that the energy storage comprises at least an energy storage unit and preferably at least a further energy storage unit; and the energy storage unit and, if preferably provided, the further energy storage unit is/are arranged radially to the coil axis at least in portions around the core. It is particularly preferable for the energy storage to completely surrounds the coil axis. The coil is preferably located between the energy storage and the core, and is preferably wound around the latter.

This arrangement of the energy storage can be an alternative to the arrangement of the energy storage described above, in which the energy storage is arranged in the direction of the coil axis relative to the core.

If the energy storage comprises multiple energy storage units, the arrangements may preferably be combined. For example, one of the energy storage units is then arranged radially to the coil axis around the core and the other of the energy storage units is arranged in the direction of the coil axis relative to the core, i.e. next to the core.

Particularly preferred is/are the core and/or the field collector and/or, if preferably provided, the further field collector made of a material with a high (material-specific) relative magnetic permeability and/or a (material-specific) saturation flux density that is as high as possible.

Examples of the material are ferrites, in particular soft magnetic ferrites, or amorphous metals such as SiFe, which is also available under the brand name ARNON, or Mu metals such as NiFe alloys.

The core and/or the field collector and/or, if preferably provided, the further field collector is/are, for example, a solid material or a layered structure made from a plurality of layers.

The layer structure is preferably made up of a large number of thin layers, such as thin films or thin sheets, between which electrically insulating layers are arranged. The electrically insulating layers can bond the thin layers to each other.

If the core has the layered structure, the individual layers have a thickness of 0.015 mm, . . . , 0.025 mm, . . . , 0.035 mm, . . . , 0.050 mm, for example. The electrically insulating layers can have the same thicknesses or be thinner.

Preferably, the core and/or the field collector and/or, if preferably provided, the further field collector is/are formed from an electrically poorly conducting material, in particular an insulator, with a high relative magnetic permeability and a saturation flux density that is as high as possible.

Preferably, the relative magnetic permeability is in a range of 100, . . . , 1000, . . . , 5000, and particularly preferably in a range of 500, . . . , 1000, . . . , 1500.

The material also preferably has a saturation flux density that is as high as possible. The saturation flux density is preferably in a range of greater than or equal to 0.3, 0.4, 0.5, 0.6, 0.7, 0.8, 0.9, 1.0, 1.1, 1.2, 1.3, 1.4, 1.5, or 1.6 Tesla, particularly preferably in a range of 0.4 to 0.7 Tesla for ferrites, or most preferably in a range of 1 to 1.5 Tesla for amorphous metals, such as SiFe.

In particular, it is preferred that said elements (core and/or field collectors) are formed from a solid material when the material is the insulator, and that said elements (core and/or field collectors) have the layered structure when the material is poor but to some degree electrically conductive.

If the core and/or the field collector and/or, if preferably provided, the further field collector is/are formed from the solid material, the material is preferably the aforementioned ferrite.

If, on the other hand, the core and/or the field collector and/or, if preferably provided, the further field collector has/have the layered structure, the material of the layers is preferably the amorphous metal, such as SiFe, or the Mu metal, such as the NiFe alloy.

Preferably, the field collector and/or the further field collector is/are a separate element from the core and made of a different material.

Because the materials available for solid material do not have the high magnetic values of amorphous metals, for example, which in turn are not suitable for solid material due to their electrical conductivity, it is particularly preferable for the core to have a layered structure of thin foils or sheets, e.g. SiFe, and for the field collectors to be made of solid material, e.g. ferrite. In this way, the field collectors can concentrate the field as far as possible independent of direction and at the same time the core can conduct the largest possible field through the coil.

The core made up of the layers preferably has a connecting portion at the end(s) facing the field collector(s) which runs transversely to the coil axis and interlocks with a recess in the corresponding field collector in order to connect the elements to one another.

The connecting portion(s) preferably has/have the same dimensions on both sides running transverse to the coil axis and is/are therefore symmetrical to the coil axis. The respective connecting potion preferably has the same overall external dimensions as the corresponding field collector and is therefore larger in cross-section than the core. In this way, the magnetic contact resistance between the core and the field collector(s) is kept low.

The connecting portion(s) of the core are an integral part of the structure respectively have the layered structure. The connecting portion(s) thus ensure that the alternating magnetic field collected by the field collector(s) is conducted through the coil. The coil is preferably located on a portion of the core that is connected to the connecting portion in the direction of the coil axis or, if the connecting portion is formed on each side, is located between the connecting portions.

The implant is preferably designed in such a way that
for recharging the energy storage, the alternating magnetic field with a flux density B₀ is to be generated as intended in the region of the implanted implant, as a result of which a corresponding charging voltage is induced in the coil, which corresponds to a change in the magnetic flux of the external alternating magnetic field bundled in the core and leads to a charging (alternating) current emitted by the coil and supplied directly or indirectly to the energy storage,
the core and/or the field collector and/or, if preferably provided, the further field collector is/are formed of the material having the high saturation flux density, and
the geometry of the core (7) and/or of the field collector and/or, if preferably provided, of the further field collector is selected such that a core flux density B_{C}, which results in the core of the coil from the multiplied flux density (n×B₀) reduced by an opposing field generated by the charging (alternating) current, is in the range, in particular plus/minus 1%, 2%, 3%, 4%, 5% or 10%, of the (material-specific) saturation flux density.

The magnetic flux density B₀ can have the values already mentioned, in particular in the range from 5 mT to 10 mT. This magnetic flux density B₀ results in a strong magnetic flux of the alternating magnetic field within the core because the field collector(s) bundle the field correspondingly strongly. According to the invention, the material is preferably selected so that the field weakened by the opposing field in the core is close to the saturation flux density or in the range mentioned.

This design makes optimum use of the externally generated alternating magnetic field.

The charging current is preferably supplied from the coil 6 to the energy storage 4 via charging electronics, which has at least one rectifier and at least one capacitor.

The electronic implant is preferably an electronic pacemaker, in particular a cardiac pacemaker, and
the electronics assembly connected with the electrode portion, is preferably configured to monitor the bodily function via the electrode portion and to generate an impulse, in particular a voltage impulse, and to emit the same via the electrode portion to the body portion to control the bodily function.

The saturation flux density mentioned at various points above refers to the flux density range specific to the material at which the corresponding magnetization characteristic curve (B-H characteristic curve) has a kink region or transition region, below which the magnetization characteristic curve is essentially linear and above which the magnetization characteristic curve runs with a lower gradient (namely with μ₀). Preferably, the saturation flux density refers to the flux density at which—with a further increase in the field strength H of the acting alternating magnetic field—the polarization of the material no longer increases.

As mentioned above, when charging the energy storage, the corresponding field vector (B vector) of the alternating electromagnetic field should preferably point in the direction of the coil axis defining the orientation of the implant in the body and pass through the coil, core and field collector(s). This leads to the best possible induction in terms of induced charging voltage and the resulting charging currents. The field collector and/or the further field collector have a significant effect, as they make the charging of the energy storage insensitive to tilting/misalignment between the field vector (B vector) and the coil axis over a wide range.

Preferably, the electronics assembly of the implant further comprises a communication unit so as to communicate with the outside (outside the body of the living being), for example to transmit setting data, setting commands, analysis data and/or information data indicating the charge status of the energy storage. A data memory is preferably provided for storing the data between communications.

Further, the electronics assembly is preferably configured to detect a gradient of the induced charging voltage and/or the charging current driven by the induced charging voltage, and to generate a signal containing information about the gradient. The communication unit sends this signal to the outside, wherein a higher-level unit receiving the signal, such as a charging device, can use the information about the gradient to infer the desired position and orientation of the implant. Knowing the position and orientation of the implant, the higher-level unit, such as the charging unit, can align the B-field vector of the alternating magnetic field accordingly to optimize/improve charging.

It should be emphasized here that the initial orientation of the B-field vector can take any direction in space, because subsequent adaptation to the position and orientation of the implant is possible. It also follows from this that no attention needs to be paid to the resulting position and alignment of the implant when the implant is implanted.

Particularly preferably, the implant according to the invention, especially the preferred variants of the energy receiving portion, is designed and dimensioned in such a way that with parallel alignment of the B-field vector and coil axis and with a magnetic flux density B₀ of 0.5 mT to 30 mT of the external alternating magnetic field, a mean magnetic flux of 0.2×10⁻⁶ to 36×10⁻⁶ Vs (Weber) is established in the core.

In case that the AC resistance (ωL) of the coil significantly exceeds the ohmic resistance R of the coil, it is preferred that the electronics assembly comprises an additional resonant capacitor and that the alternating magnetic field is generated at the frequency resulting from the values of the coil, the resonant capacitor, the ohmic resistance and the load, i.e. essentially the ohmic resistance R limits the size/intensity of the charging current.

Finally, the structure of the implant is preferably optimized in terms of the charging current achieved with a predetermined size of the implant, which is given by the volume of the housing, e.g. cylindrical housing, with a coil with W windings and a winding cross-section A and an external magnetic field B₀. In the case of (ωL>>R), the first approximation is that the charging current is proportional to the ratio of the magnetic flux through the coil to the inductance of the coil (Φ/L), in the resonance case (Φ/R). Therefore, the design ratio of the sum of the dimensions of the field collectors in the direction of the coil axis to the length of the core in connection with the coil wound over the core is an important measure of the amount of charge current that can be drawn. The parameters mentioned influence both the magnetic flux in the core and the inductance of the coil. In the implant according to the invention, the design ratio is realized in such a way that the charging current reaches its maximum or is at most 10% lower. In connection with the charging current achieved, it should be emphasized that the ohmic resistance R of the coil may be kept very low with a simultaneously high field strength in the core, compared to a core with a constant diameter corresponding to the field collectors. The lower ohmic resistance generates lower losses and therefore results in significantly lower heat generation. Due to the intended location of the implant in the human body, e.g. heart, brain, tissue, vessel or organ, this is a very important factor.

The following can be deduced from the explanations of the implant according to the invention and its preferred features:

The described design of the implant, in particular the energy receiving portion, opens up the significant possibility of finding an optimum for the respective application of the implant, for example as a cardiac pacemaker, brain pacemaker, organ pacemaker or analysis unit, by means of many variable parameters. This optimum can be found by maximizing the magnetic field in the core, minimizing the weight and losses of the coil and, as a first approximation, specifying the volume of the implant by the dimensions of the magnetic components.

With a desired small size and weight and an autonomous running time of the implant of, for example, one year and a reasonable charging time of, for example, less than 30 minutes, the described design of the implant, in particular the energy receiving portion, is optimized in terms of volume and weight in a first approximation by the largest possible external magnetic field (5-20 mT) and in a second approximation by the losses or heating that occur during charging and the field concentration in the core.

The above explanations apply equally to the following embodiment.

Below, a preferred embodiment is explained with reference to the attached figures.

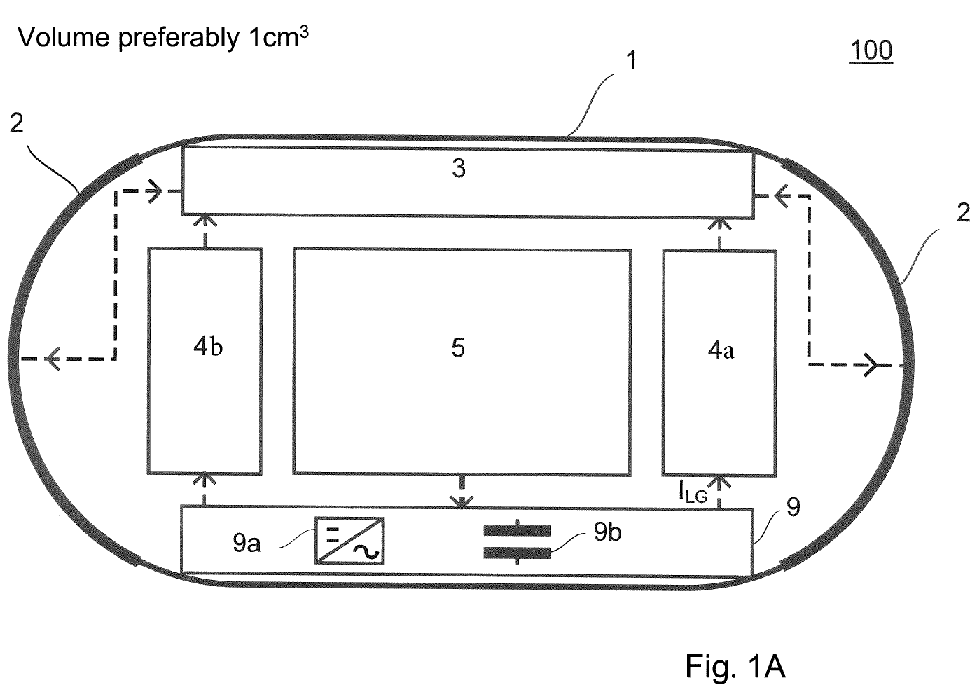

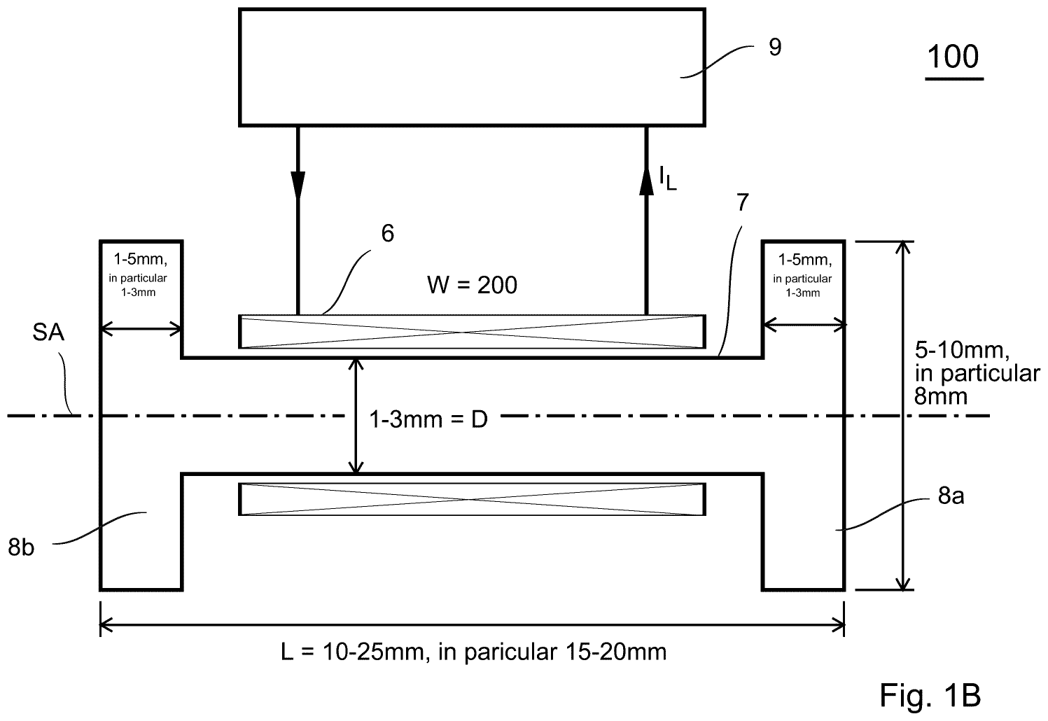

FIG. 1A shows a preferred embodiment of the implant according to the invention, wherein the illustration is only schematic;

FIG. 1B shows a schematic sectional view of an energy receiving portion of the implant according to the invention; and

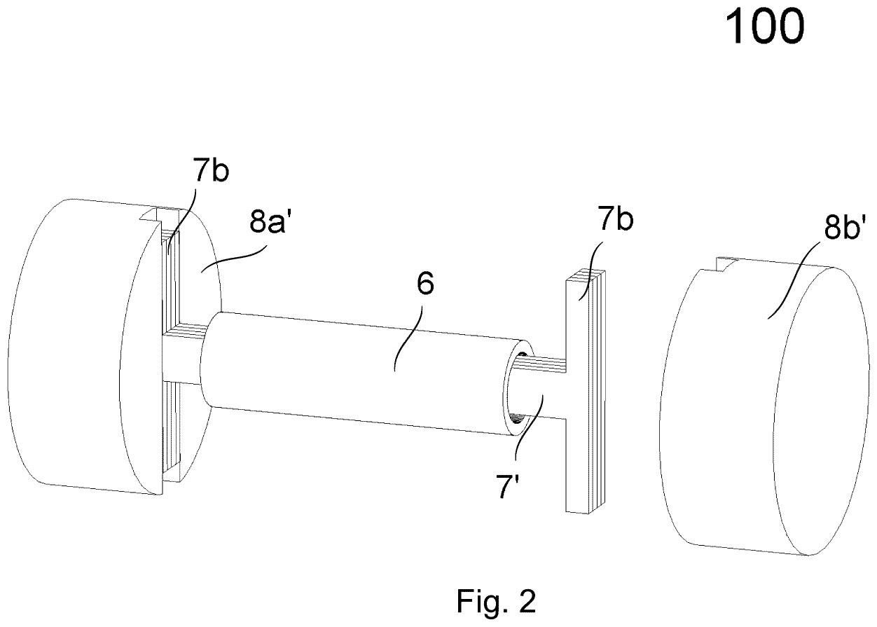

FIG. 2 shows a preferred variant of the energy receiving portion of the implant according to the invention.

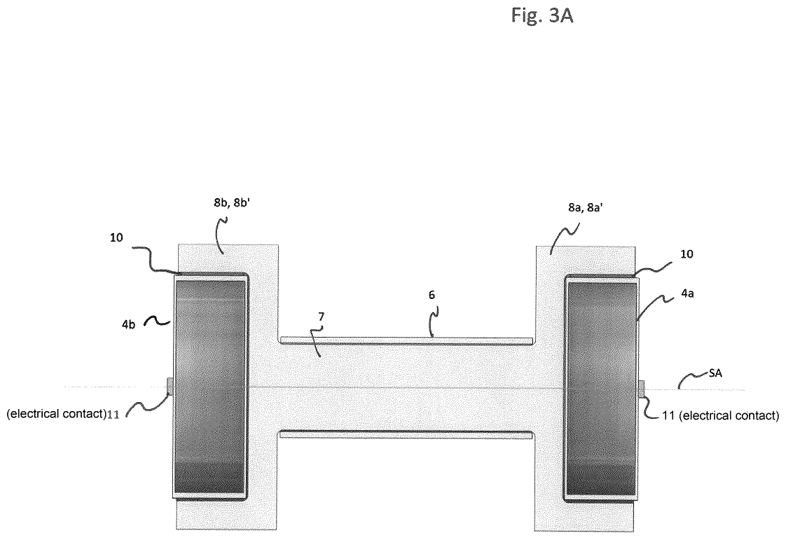

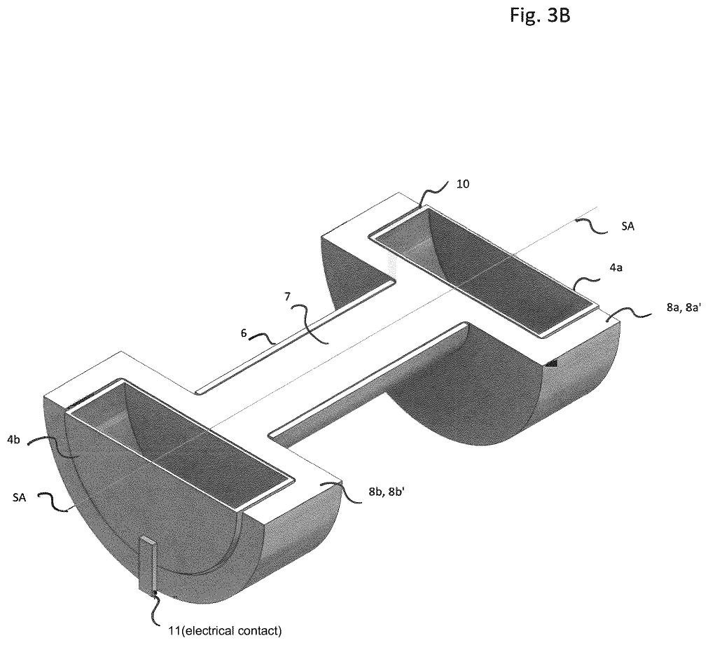

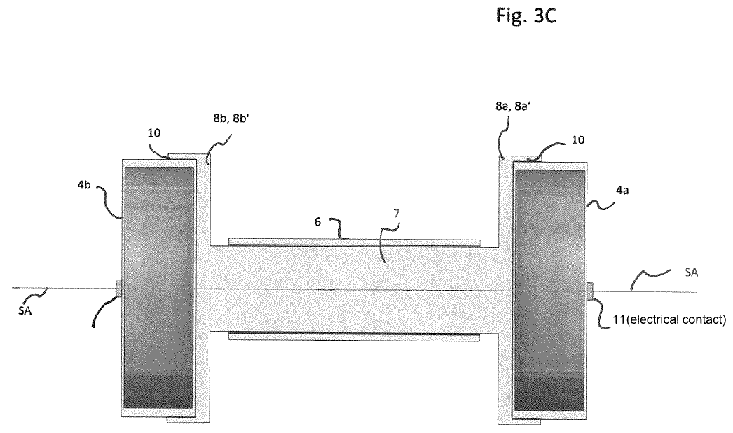

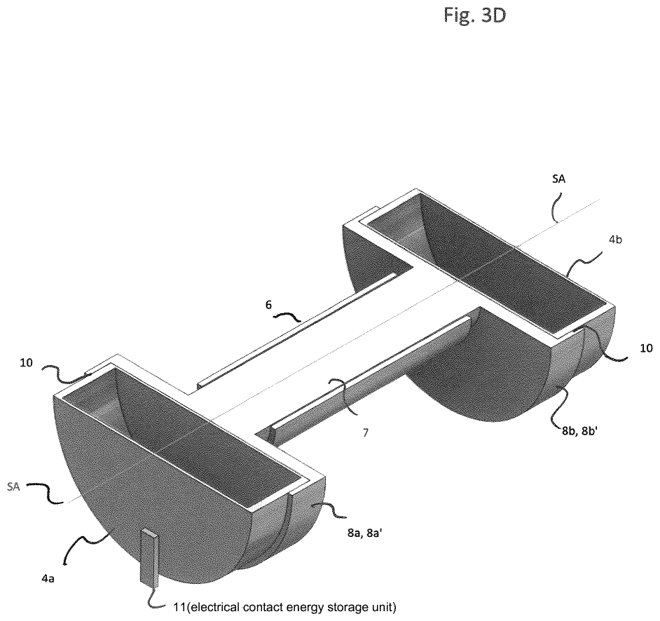

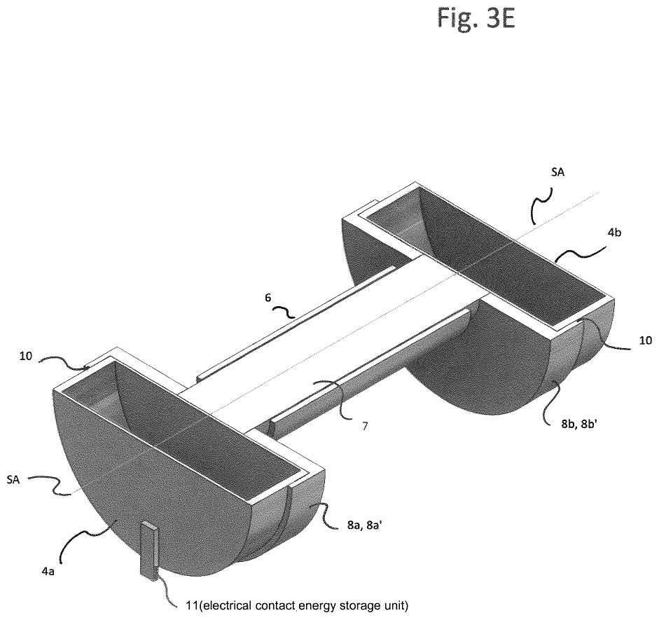

FIGS. 3A to 3D show preferred configurations of the field collector(s), wherein the core and field collectors are monolithic and FIG. 3E shows an alternative variant of the field collector(s) as separate elements. FIGS. 3A to 3E only show the casing of the energy storage without electrochemical content.

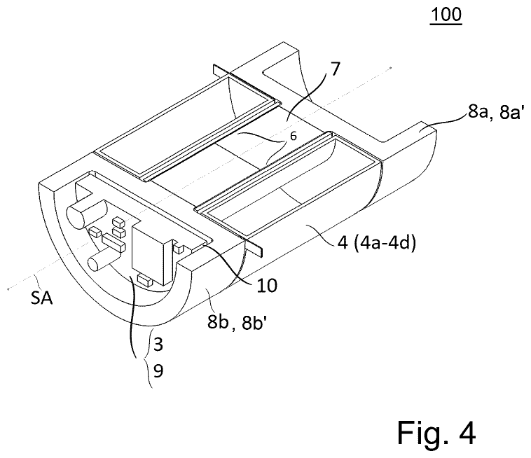

FIG. 4 shows an alternative variant of the implant, wherein the core and field collectors are built up identically to FIGS. 3A and 3B, and the energy storage comprises an annular housing adapted to the core. FIG. 4 shows only the casing of the housing without electrochemical contents.

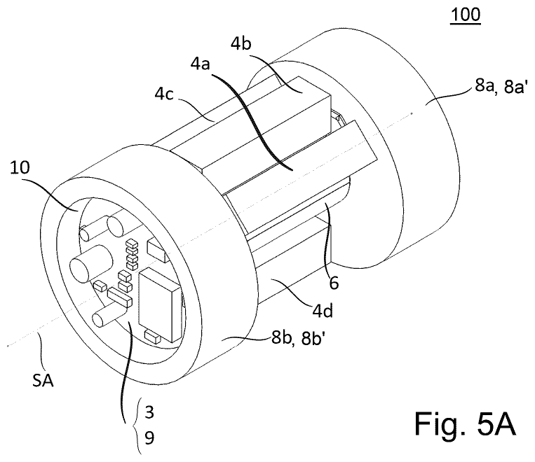

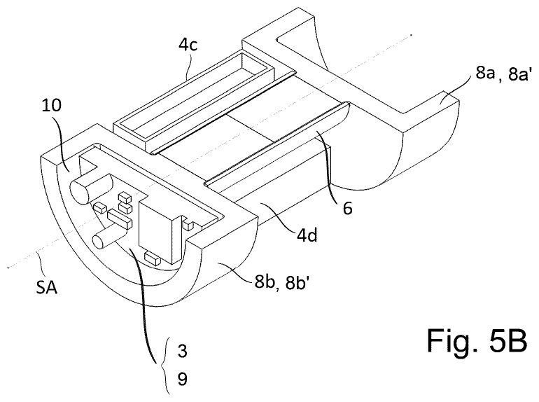

FIGS. 5A and 5B show a further alternative variant of the implant, wherein the core and field collectors are built up identically to FIGS. 3A and 3B, and energy storage units are arranged around the core. FIG. 5B shows only the casing of the housing of the energy storage unit without electrochemical content.

FIG. 1A schematically shows the configuration of an implant 100 according to the invention.

The implant 100 is preferably implanted completely into a human body. The implant 100 is, for example, a cardiac pacemaker, a brain pacemaker, an organ pacemaker or an analysis unit. The latter analysis unit is, for example, configured to determine parameters such as blood pressure and/or blood values continuously or at certain intervals. The implant is particularly preferably a pacemaker or pacemaker network which is located in the human heart or on the human heart or is to be implanted into these positions.

The implant 100 preferably has a housing 1 that accommodates all elements of the implant 100 and is preferably hermetically encapsulated. The housing 1 is made of titanium or glass, for example.

The implant 100 has an electrode portion with electrodes 2, which comprises a certain number of electrodes 2 depending on the purpose of the implant or which bodily function it is intended to monitor/stimulate. The electrodes 2 are, in accordance with the intended use, connected to or in contact with the body portion, for example the heart or the brain, that is to be monitored and/or to be stimulated.

The electrodes 2 may comprise, for example at their ends, spiral segments which are twisted into the body portion and thus anchored in it. One of the electrodes and/or the housing, if conductive, can serve as a ground electrode.

In general, the electronic pacemaker or the pacemaker network according to the invention may be a cardiac pacemaker according to any NBG code.

In general, the electrodes mentioned can be, for example, cable electrodes or electrode surfaces exposed on the outer surface.

The housing 1 also accommodates an electronics assembly 3, which is configured to monitor and/or stimulate a bodily function via the electrodes 2, and an energy storage with at least one, preferably two, energy storage units 4a, 4b, which supply the electronics assembly 3 with electrical energy, as well as charging electronics 9. Preferably, the charging electronics 9 comprises a rectifier 9a and a capacitor 9b, which rectify a charging (alternating) current (AC) I_{L} emitted by the coil 6 and supply it to the energy storage units 4 as I_{LG}, in that the rectifier 9a rectifies the charging (alternating) current (AC) I_{L} emitted by the coil 6 and supplies it to the capacitor 9_{b}, and the capacitor 9_{b} then passes the current I_{LG} on to the energy storage units 4.

The energy storage units, i.e. the one energy storage unit 4a and the other energy storage unit 4b, are preferably each rechargeable, electrochemical accumulators, for example lithium-ion accumulators, which supply the entire implant 100 with electrical energy for, for example, 0.5 to 1.5 years before they have to be recharged.

The energy storage units 4a, 4b may be recharged without contact, using induction. For this purpose, the implant 100 has an energy receiving portion 5, which is an essential element of the invention.

FIG. 1B shows a longitudinal section of the energy receiving portion 5 according to the invention.

This comprises a coil 6 with, for example, 200 windings (W=200).

The coil 6 is wound on and around a core 7, which extends along a coil axis SA. The coil axis SA also corresponds to a longitudinal axis of the implant 100 respectively the housing 1.

A field collector 8a and a further field collector 8b are located at the respective ends of the coil 6 and the core 7 respectively, the dimensions of which are larger transverse to the coil axis SA than those of the core 7 within the coil 6. A diameter of the core 7 within the coil 6 measured perpendicular to the coil axis SA is preferably 1 mm (millimeter) in FIG. 1A. Consequently, the coil 6 wound on this also has an internal diameter of 1 mm. The stated diameters of the core 7 or inner diameter of the coil 6 can range from 1 mm to 3 mm. A length of the coil 6 preferably corresponds to the length of the core 7 between the field collectors 8a, 8b. In other words, the ends of the coil 6 preferably correspond to the ends of the core 7.

The dimensions of the field collector 8a and the other field collector 8b are much larger. The corresponding diameters measured perpendicular to the coil axis SA are, for example, 5 mm to 10 mm, preferably 8 mm, and thus have 64 times the cross-sectional area of the core 7 with a diameter of 1 mm of the core 7 and a diameter of 8 mm of the field collectors 8a, 8b.

The core 6 and both field collectors 8a, 8b preferably have a circular cross-section perpendicular to the coil axis SA. Alternatively, the cross-section can also be rectangular, in particular square.

In conjunction with FIGS. 1A and 1B, it is understandable that the one energy storage unit 4a of the energy storage 4 is arranged in the direction of the coil axis SA relative to the core 7 next to the field collector 8a, and that the other energy storage unit 4b is arranged in the direction of the coil axis SA next to the other field collector 8b. This relative arrangement gives the entire implant a very compact structure.

A length L of the core 7 with the field collectors 8a, 8b can be 10 mm to 25 mm, in particular 15 mm to 20 mm, with dimensions of the field collector 8a and the further field collector 8b in the direction of the coil axis being 1 to 5 mm.

The invention is not limited to the dimensions mentioned. These are merely examples.

The field collector 8a and the further field collector 8b can be elements separate from the core 7 or integral components of the core 7. The latter is shown in FIG. 1B. Both field collectors 8a, 8b are monolithic with the core made of a uniform material. The material is a ferrite, for example. The monolithic structure is particularly preferable if the material is an insulator or at least a material with poor electrical conductivity, for example a ferrite, because no or hardly any eddy currents occur.

In general, the core and/or the field collectors 8a, 8b are formed from a material with a high relative magnetic permeability μᵣ (particularly preferably in a range of 1000), with the highest possible saturation flux density (for example 0.4 to 0.7 Tesla for ferrites; or 1 to 1.5 Tesla for the amorphous metals mentioned with reference to FIG. 2 below, such as SiFe) and the lowest possible electrical conductivity, preferably an insulator.

An idea essential to the invention is to form the field collectors 8a, 8b perpendicular to the coil axis SA larger than the core 6 in such a way that the energy storage units 4a, 4b of the implant 100 can be charged by induction even at low frequencies (e.g. 2 kHz), which are otherwise not used in inductive contactless energy transmission.

This is illustrated by the following example, to which the invention is not limited.

When the energy storage units 4a, 4b of the implant 100 need to be charged, an alternating magnetic field with a magnetic flux density (B field) B₀ of approximately 5 mT (milli Tesla), which is homogeneous over a wide area including the implant 100, is generated by a charging device not shown. In particular, the field is preferably aligned in the direction of the coil axis SA (B vector) and passes through the coil 6.

Strictly speaking, the alternating magnetic field is an alternating electromagnetic field. However, the electrical component of this field is of secondary importance, which is why only the alternating magnetic field is referred to in this application. However, a pure alternating magnetic field is also covered by the invention.

The frequency f of the alternating magnetic field is in the range of 2 kHz, for example. At these low frequencies, the alternating magnetic field penetrates well and deeply into human tissue, for example up to the human heart, where the implant 100 is preferably located.

Because the energy receiving portion 5 has the aforementioned core 7 with the field collectors 8a, 8b, the core 7 receives sufficient field so that the coil 6 generates a sufficiently high charging (alternating) current (AC) I_{L} to charge the energy storage units 4a, 4b. Due to the dimensions perpendicular to the coil axis SA of the field collector 8a and the other field collector 8b, there is an increased core flux density B_{C} within the core 7. For example, the core flux density B_{C} exceeds the magnetic flux density B₀ by up to 200 times (B_{C}=200B₀).

If the magnetic flux density B₀ of the alternating magnetic field generated by the charging device, which is present in the area of the implant, is 5 mT, the core flux density B_{C} in the unloaded state is therefore approximately 1 T. However, the aforementioned core flux density B_{C} is reduced by the opposing field occurring within the coil 6, which is caused by the charging (alternating) current (AC) I_{L}.

The resulting core flux density B_{C} then amounts to a total of approximately 0.6 T, which results in a high induced voltage (approx. 15 V in the embodiment) and a high average charging current I_{LG} (approx. 200 mA in the embodiment). The coil 6 shown has an inductance of approximately 2 mH.

These values allow the energy storage units 4a, 4b to be charged in approximately 30 min with a charge of approximately 400 coulombs.

Irrespective of the example described, the ratio of the diameter of the field collectors 8a, 8b to the diameter of the core 7 is selected to be larger the weaker the flux density B₀ and/or the lower the frequency f of the alternating magnetic field generated for charging. With this in mind, the dimensions of the core 6, the field collectors 8a, 8b, the parameters of the coil 6 and the remaining elements are selected so that the weight of the entire implant 100 is low and lies in the range from 1.5 g to 2.5 g, preferably 2 g (grams), particularly preferably less than 2 g.

The charging current is supplied from the coil 6 to the energy storage respectively the energy storage units 4a, 4b, preferably via the shown charging electronics 9.

It is clear and understandable from the above that the core 7 and the field collectors 8a, 8b are made of a material that has a high relative magnetic permeability μᵣ with the highest possible saturation flux density. In the above example, the core 6 and the field collectors 8a, 8b, which are formed monolithically from a uniform material (ferrite), had a saturation flux density of approximately 0.6 T. To increase this further, a different material may be used.

An amorphous metal, for example SiFe, can be used as a particularly preferred alternative material for the core 7 and/or the field collectors 8a, 8b. This type of metal is available on the market under the brand name ARNON, for example.

FIG. 2 shows in principle a preferred structure of an alternative core 7′ including alternative field collectors 8a′, 8b′ of the implant 100. The core 7′ has a structure built up from of a large number of thin metal layers, such as thin sheet metal layers or thin metal foils, which are separated from each other by insulating layers. The metal layers are preferably formed from amorphous metal, for example SiFe. The field collector 8a′ and the other field collector 8b′, on the other hand, are made of solid material, for example ferrite.

All other elements are identical to those shown in FIGS. 1A and 1B, which is why reference is made to the corresponding explanations.

Since the alternative material, the amorphous metal, is not a solid material due to the eddy currents, the elements mentioned have the layered structure.

The thickness of each individual layer perpendicular to the coil axis SA′ shown is in the order of 0.015 mm to 0.050 mm, particularly preferably 0.025 mm. This structure keeps eddy currents to a minimum. Core 7′ and field collectors 8a′, 8b′ preferably have a rectangular cross-section perpendicular to the coil axis SA′.

The structure of the core 7′ shown in FIG. 2 made of the aforementioned amorphous metal (SiFe) has a saturation flux density of 1 T to 1.5 T and thus allows, for example, an increase in the magnetic flux density B₀ of the alternating magnetic field generated for charging and a reduction in the charging time. This also allows a small design to be achieved.

However, as the magnetic flux transverse to the layering is not as high as along the layers due to the insulating layers, such a structure of the field collectors is not as effective as with solid material. On the other hand, the flux density is generally lower in the field collectors due to the larger volumes, so that a structure made of solid material is more suitable. The structure shown in FIG. 2 therefore corresponds to the preferred variant, in which the core 7′ and the collectors 8a′, 8b′ are made of different materials.

At the transitions between the materials, suitable constructions with corresponding surface designs are applied to ensure that the magnetic resistance remains low. One possible construction is shown in FIG. 2 and is realized as an example by connecting portions 7a, 7b, which are an integral part of the core 7′ and thus of the layered structure. The connecting portions 7a′, 7b′, which extend transversely to the coil axis SA′, interlock with a respective recess 10 in the corresponding field collector 8a′, 8b′ in order to connect the elements shown with one another.

The coil 6 is preferably seated on a portion of the core 7′ which is adjacent to the connecting sections 7a, 7b in the direction of the coil axis SA′ or which is located between the connecting portions 7a, 7b.

The connecting portions 7a, 7b are each symmetrical to the coil axis SA′ and have the same dimensions perpendicular to the coil axis SA′ as the respective field collectors 8a′, 8b′. In the direction of the coil axis SA′, the dimensions of the connecting portions 7a, 7b are preferably equal to the depth of the recesses in the field collectors 8a′, 8b′, for example 1 mm.

FIGS. 3A to 3D show alternative configurations for the field collectors. In these figures, the field collectors are a monolithic part of the core 7, as already explained with reference to FIG. 1B. However, the alternative configurations of the field collectors shown in FIGS. 3A to 3D are not limited to this monolithic configuration, but can also be realized as separate elements, as in the variant explained with reference to FIG. 2. This is preferably the case if core 7 and the field collector(s) 8a, 8a′, 8b, 8b′ are made of different materials. To illustrate that the field collector(s) 8a, 8a′, 8b, 8b′ may be monolithic or separate elements, the field collectors in FIGS. 3A to 3D bear the reference signs already used in FIGS. 1B and 2.

FIGS. 3A to 3D show sectional views of a longitudinal section, with the coil axis SA of the coil 6 lying in the corresponding resulting sectional planes.

As can be seen from FIGS. 3A to 3D, the field collectors, i.e. the field collector 8a, 8a′ and the further field collector 8b, 8b′, each have a recess 10 and preferably have a uniform wall thickness starting from the core 7 to the end face pointing in the direction of the coil axis SA.

The energy storage units 4a, 4b are each inserted into the recesses 10.

In the configuration shown in FIGS. 3A and 3B, the recesses 10 are dimensioned in such a way that they almost completely accommodate the respective energy storage unit 4a, 4b used. This means that the housing of the respective energy storage unit 4a, 4b hardly influences/impairs the guidance of the alternating magnetic field, because the alternating magnetic field mainly enters at the end faces facing towards the direction of the coil axis SA and the outer surfaces of the field collectors 8a, 8a′, 8b, 8b′ parallel to the coil axis SA.

The configuration shown in FIGS. 3C and 3D shows an alternative configuration of the field collectors 8a, 8a′, 8b, 8b′. This differs from that shown in FIGS. 3A and 3B in that the recesses 10 have smaller dimensions (depth) in the direction of the coil axis SA than in FIGS. 3A and 3B. In addition, portions of the field collector(s) 8a, 8a′, 8b, 8b′ that run in the direction of the coil axis SA are thinner than sections that run perpendicular to the coil axis SA.

The dimensioning of the recesses 10 according to FIGS. 3C and 3D means that the recesses 10 do not completely accommodate the energy storage units 4a, 4b, but that the housings of the energy storage units 4a, 4b protrude in the direction of the coil axes SA with respect to the respective field collector 8a, 8a′, 8b, 8b′.

In this case, the housings of the energy storage units 4a, 4b are preferably made of a material that at least partly assumes the function of the field collectors 8a, 8a′, 8b, 8b′. For example, the housings of the energy storage units 4a, 4b are made of the materials already mentioned in connection with the core 7, 7′ or the field collectors 8a, 8a′, 8b, 8b′ (ferrite, amorphous metal, e.g. SiFe, or mu-metal, e.g. NiFe).

In another variant according to FIG. 3E, the field collector 8a, 8a′ and/or the further field collector 8b, 8b′ can be realized exclusively by the housing of the respective energy storage unit 4a, 4b. In this case, the housings of the respective energy storage unit 4a, 4b are again preferably made of the materials mentioned (ferrite, amorphous metal, e.g. SiFe, or mu-metal, e.g. NiFe). In this other variant, the field collectors 8a, 8a′, 8b, 8b′ are not collectors in the strict sense, but components of the same shape as in FIG. 3D, which in this case only serve to hold or center the energy storage units and can also be made of a non-magnetic material, e.g. plastic.

The reference sign 11 indicates an electrical contact of the energy storage units 4a, 4b.

FIG. 4 shows a preferred alternative variant of the implant 100 according to the invention.

The energy receiving portion 5 of the present variant of the implant 100 corresponds to that shown in FIGS. 3A and 3B, with the field collectors 8a, 8a′, 8b, 8b′ additionally having the recesses 10 described above.

The implant 100 shown in FIG. 4 differs from the implant 100 described with reference to FIGS. 1A, 1B, 3A and 3B above in that one arrangement of the energy storage 4 and the electronics 3, 9 are interchanged.

In the variant shown in FIG. 4, the recesses 10 are dimensioned in such a way that the electronics assembly 3 and the charging electronics 9 are completely accommodated in one of the recesses 10. Alternatively, parts of the electronics assembly 3 and/or the charging electronics 9 can be divided between the two recesses 10. The alternating magnetic field mainly occurs at the end faces facing towards the direction of the coil axis SA and the outer faces of the field collectors 8a, 8a′, 8b, 8b′, so that the electronics assembly 3 and/or the charging electronics 9 hardly influence the alternating magnetic field.

The energy storage 4 preferably has a single housing being adapted to the outer contour or outer surface of the coil 6. The core 7 and the coil 6 preferably have a circular cross-section perpendicular to the coil axis SA. As a result, the inner surface or the surface of the housing of the energy storage 4 facing the coil 6 is annular in cross-section (transverse to the coil axis SA). FIG. 4 only shows the housing or casing of the energy storage 4 and does not show the electrochemical content.

According to FIG. 4, the energy storage 4 is accommodated in a single housing that completely surrounds the core 7. For example, the housing of the energy storage 4 can be wrapped or bent around the core 7 or the coil 6 for this arrangement. Alternatively, the housing of the energy storage 4 can preferably be wound or bent in such a way that the energy storage 4 only partially surround the core 7 or the coil 6.

Alternatively, the energy storage 4 can be made up of a large number of energy storage units, each of which has a housing that corresponds to a segment of a circle around the core 7. When assembled flush, the energy storage units then completely or partially surround the core 7.

In the event that the energy storage 4 or the energy storage units only partially surround the core 7 or the coil 6, the free space may be used, for example, for parts of the electronics assembly 3 and/or the charging electronics 9 and/or a communication unit for communication with the outside or another implant.

The housing of the energy storage 4 or the energy storage composed of the energy storage units has an axis of symmetry that is preferably identical to the coil axis SA.

Preferably, the dimensions of the energy storage 4 or the energy storage units are selected radially to the coil axis in such a way that a surface facing away from the coil axis SA is flush with the field collectors 8a, 8a′, 8b, 8b′.

FIGS. 5A and 5B show a further preferred alternative variant of the implant 100 according to the invention, which differs from that in FIG. 4 only in the design of the energy storage 4.

The energy storage 4 has a plurality of energy storage units 4a, 4b, 4c, 4d, which are arranged radially to the coil axis SA around the core 7 or the coil 6 wound thereon, each of the energy storage units 4a, 4b, 4c, 4d, having an independent housing.

The housings each have a cuboid shape whose longitudinal extension runs parallel to the coil axis SA. The length of the housing in this direction, i.e. parallel to the coil axis SA, corresponds to the length of the core 7 or the distance between the field collectors 8a, 8b.

The energy storage units 4a, 4b, 4c, 4d, are arranged around the core 7 at distances from each other in the circumferential direction.

The number of energy storage units 4a, 4b, 4c, 4d, can be selected so that together they completely surround the core 7 or, as shown in FIG. 5A, only surround it in sections/partially.

The free space shown in FIG. 5A—which is not occupied by any of the energy storage units 4a, 4b, 4c, 4d,—may be used, for example, for parts of the electronics assembly 3 and/or the charging electronics 9 and/or a communication unit for communication with the outside or another implant.

The arrangements according to FIGS. 4, 5A and 5B may also be realized with a core 7 and field collectors 8a′, 8b′ (FIG. 2), wherein a recess as in FIG. 3 can also be formed in the field collector(s) 8a′, 8b′ if required.

The implant 100 may be part of said pacemaker network having a plurality of such implants 100 in which corresponding elements as explained with reference to FIGS. 1A, 1B and/or 2 and/or 3A to 3D are included. In terms of weight, each of these implants weighs approximately 2 g. The explanations before the description of the figures apply accordingly to the embodiment and vice versa.

1. Electronic implant for implantation into a body of a living being and for monitoring a bodily function, comprising a pacemaker for monitoring and controlling the bodily function, the implant comprising:
an electrode portion that is, according to its intended purpose, to be attached to or to be arranged at a body portion;
an electronics assembly connected to the electrode portion, which is configured to monitor at least the bodily function via the electrode portion;
an energy storage to supply the electronics assembly with electrical energy which can be recharged with electrical energy after discharge; and
an energy receiving portion electrically connected to the energy storage, which is configured so as to be able to receive energy without contact and to deliver the energy to the energy storage for recharging the energy storage;
wherein
the energy receiving portion comprises:
a coil extending along a coil axis and being configured to receive the energy and to deliver the energy to the energy storage when passed through by an external alternating magnetic field,
a core, which is located in the coil and extends along the coil axis, and
at least one field collector, which is located at one end of the core in the direction of the coil axis and has larger dimensions transverse to the coil axis than the core.

2. The electronic implant according to claim 1, wherein
the field collector is a part of the core, being monolithic with the core and is formed from the same material.

3. The electronic implant according to claim 1, further comprising:
a further field collector, which is located in the direction of the coil axis at another end of the core and has larger dimensions transverse to the coil axis than the core.

4. The electronic implant according to claim 3, wherein
the further field collector is a part of the core, being monolithic with the core and is formed from the same material.

5. The electronic implant according to claim 1, wherein
the field collector and/or the further field collector is a separate element from the core and is formed from a different material.

6. The electronic implant according to claim 1, wherein
the energy storage comprises at least an energy storage unit and at least a further energy storage unit; and
the energy storage unit and the further energy storage unit is/are arranged in the direction of the coil axis relative to the core.

7. The electronic implant according to claim 5, wherein
the energy storage comprises at least an energy storage unit and at least a further energy storage unit; and
the energy storage unit and the further energy storage unit each have a housing which acts as the field collector and/or the further field collector.

8. The electronic implant according to claim 1, wherein
the energy storage comprises at least an energy storage unit and at least a further energy storage unit, and
the field collector and the further field collector has/have a recess in which the energy storage unit and the further energy storage unit is/are accommodated.

9. The electronic implant according to claim 3, wherein
the coil is wound on and around the core between the field collector and the further field collector.

10. The electronic implant according to claim 1, wherein
the energy storage comprises at least an energy storage unit and at least a further energy storage unit; and
the energy storage unit and the further energy storage unit(s) is/are arranged radially to the coil axis at least in portions around the coil.

11. The electronic implant according to claim 10, wherein
the energy storage completely surrounds the coil axis.

12. The electronic implant according to claim 10, wherein
the energy storage unit is arranged radially to the coil axis around the coil.

13. The electronic implant according to claim 1, wherein the core and/or the field collector and/or the further field collector is/are formed from a material with a high relative magnetic permeability and/or a saturation flux density that is as high as possible.

14. The electronic implant according to claim 1, wherein the core and/or the field collector and/or the further field collector has/have a structure formed of a plurality of individual thin layers, and
the material of these layers has a high relative magnetic permeability and/or a high saturation flux density, and is an amorphous metal.

15. The electronic implant according to claim 14, wherein
the field collector and the further field collector are an element separate from the core and are formed from a different material, and
the field collector and the further field collector are formed from the solid material and the core has the structure with the thin layers.

16. The electronic implant according to claim 1, wherein the energy receiving portion comprises at least one rectifier and at least one capacitor located between the coil and the energy storage, and
the coil transfers the received energy to the energy storage via the rectifier and the capacitor.

17. The electronic implant according to claim 1, wherein
for recharging the energy storage, the alternating magnetic field with a flux density is to be generated as intended in the area of the implanted implant, as a result of which a corresponding charging voltage is induced in the coil, which leads to a charging current emitted by the coil and supplied directly or indirectly to the energy storage,
the core and/or the field collector and/or the further field collector is/are formed of a material having a high saturation flux density, and
the geometry of the core and/or of the field collector and/or of the further field collector is selected such that a flux density BC, which results in the core of the coil from the multiplied flux density reduced by an opposing field generated by the charging (alternating) current, is in the range of the saturation flux density.

18. The Electronic implant according to claim 1, wherein the implant is an electronic pace-maker, comprising a cardiac pacemaker, and
the electronics assembly connected to the electrode portion is configured to monitor the bodily function via the electrode portion and to generate a pulse, comprising a voltage pulse, and to emit this via the electrode portion to the body portion for controlling the bodily function, and to measure, store and transmit further body data.

19. The Electronic implant according to claim 1, wherein an average magnetic flux of 0.2×10−6 to 36×10−6 Vs (Weber) is established in the core when using the alternating magnetic field with a flux density B0 of 0.5 mT to 30 mT.

20. The Electronic implant according to claim 1, wherein
an alternating current resistance (ωL) of the coil resulting from the inductance of the coil and the frequency of the external alternating magnetic field exceeds the ohmic resistance of the coil,
the electronics have a resonant capacitor, and
AC resistance and ohmic resistance for the alternating magnetic field to be used as intended are dimensioned in such a way that the coil and the resonant capacitor are in resonance.
